(19) United States
(12) Patent Application Publication
(10) Pub. No.: US 2019/0031579 A1
(43) Pub. Date: Jan. 31, 2019

(54) METHOD FOR PRODUCING ALKYL SUBSTITUTED BENZENE

(71) Applicant: FAR EASTERN NEW CENTURY CORPORATION, Taipei City (TW)

(72) Inventors: Shu-Wei Liu, Taipei City (TW); Ruey-Fen Liao, Taipei City (TW); Xin-An Lu, Taipei City (TW)

(21) Appl. No.: 15/824,359

(22) Filed: Nov. 28, 2017

(30) Foreign Application Priority Data
Jul. 28, 2017 (TW) 106125473

(51) Int. Cl. C07C 2/86; B01J 31/02

(52) U.S. Cl. CPC C07C 2/865; C07C 2/867; B01J 31/0232; B01J 2531/004; C07C 2531/12; B01J 2231/324; C07C 2531/025

(57) ABSTRACT

A method for producing alkyl substituted benzene includes (a) providing a starting material selecting from the group consisting of furan, an alkyl substituted furan, 2-methylfuran, 2,3-dimethylfuran, 2,4-dimethylfuran, 2,5-dimethylfuran, 2,5-hexanedione, and combinations thereof, and (b) subjecting the starting material to a cycloaddition reaction with a monoene in the absence of solvent and in the presence of the metal triflate catalyst to produce an alkyl substituted benzene.

### CROSS-REFERENCE TO RELATED APPLICATION

This application claims priority from Taiwanese patent application no. 106125473, filed on Jul. 28, 2017.

### FIELD

The disclosure relates to a method for producing alkyl substituted benzene, more particularly to a method for producing para-xylene without using a solvent.

### BACKGROUND

U.S. Pat. No. 9,260,359 B2 discloses a method to produce xylene, toluene, or other compounds from 2,5-dimethylfuran (DMF) and ethylene in the presence of an acid, such as a Lewis acid. A cycloaddition reaction between DMF and ethylene may be performed either with or without a solvent present . The Lewis acid may be AlCl₃ , Bi(OTf)₃, CuCl₂, Cu(Of)₂, CoCl₂, CrCl₃, Fe(OTf)₂, Gd(OTf)₃, InCl₃, In(OTf)₃, NiCl₂, Ni(OTf)₂, MnCl₂, SnCl₂, TiCl₄, VCl₂, Y(OTf)₃, P₂O₅, acetic anhydride, acetic acid, chloroacetic anhydride, and so on. In U.S. Pat. No. 9,260,359 B2, when the reaction is performed without a solvent, only acetic anhydride, acetic acid, chloroacetic anhydride, and chloroacetic acid are used as the Lewis acid, and the conversion of the starting material (DMF) into the product (para-xylene) is relatively low.

U.S. patent publication no. 2014/0296600 A1 provides a renewable route to para-xylene via cycloaddition of ethylene and 2,5-dimethylfuran and subsequent dehydration with high selectivity and high yields using acetic heterogeneous catalysts and a solvent for 2,5-dimethylfuran. The acetic heterogeneous catalysts may be a zeolite molecular sieve, activated carbon, silica, alumina, a non-zeolitic molecular sieve, and so on.

U.S. Pat. No. 8,889,938 B2 discloses methods for producing para-xylene by reacting ethylene with 2,5-hexanedione using a Lewis acid catalyst which may be copper chloride, copper triflate, yttrium triflate, a heteropolyacid, or η²-ethylene-copper(II)triflate. In all of the examples in U.S. Pat. No. 8,889,938 B2, 2,5-hexanedione or 2,5-dimethylfuran is reacted with ethylene in the presence of solvent.

U.S. Pat. No. 8,314,267 B2 discloses a method for producing para-xylene via cycloaddition of ethylene and 2,5-dimethylfuran (DMF) using catalysts which may be ZnCl₂, rare-earth exchanged Y zeolite (RE-Y), activated carbon, silica gel, and γ-alumina. The conversion of DMF to para-xylene is not satisfied.

U.S. patent publication no. 2016/0115113 A1 discloses a dimethylterephthalate production process which includes reacting substituted furan with ethylene under cycloaddition reaction conditions and in the presence of a cycloaddition catalyst to produce a bicyclic ether; dehydrating the bicyclic ether to produce a substituted phenyl; dissolving the substituted phenyl in methanol; and oxidizing and esterifying the substituted phenyl in the presence of an oxidative esterification catalyst to form dimethylterephthalate.

Please note that in the prior documents above, the conventional processes/methods for making para-xylene from renewable sources (e.g., cellulose) in the absence of a solvent and in the presence of a catalyst have relatively low conversion and selectivity and are undesirable for mass production.

### SUMMARY

Therefore, an object of the disclosure is to provide a method for producing alkyl substituted benzene, which is performed under solventless condition, and thus is performed at low cost and is environmentally friendly. By virtue of the method, alkyl substituted benzene, especially para-xylene, can be produced in good yield.

According to the disclosure, a method for producing alkyl substituted benzene includes the steps of:

(a) providing a starting material selecting from the group consisting of furan, an alkyl substituted furan, 2-methylfuran, 2,3-dimethylfuran, 2,4-dimethylfuran, 2,5-dimethylfuran, 2,5-hexanedione, and combinations thereof; and

(b) subjecting the starting material to a cycloaddition reaction with a monoene in the absence of solvent and in the presence of the metal triflate catalyst to produce an alkyl substituted benzene.

### DETAILED DESCRIPTION

A method for producing alkyl substituted benzene according an embodiment of the disclosure includes steps (a) and (b).

In step (a), a starting material is provided. The starting material is selected from the group consisting of furan, an alkyl substituted furan, 2,5-hexanedione (HD), and combinations thereof.

The alkyl substituted furan may include one or more C1 to C8 linear alkyl substituted furan. Preferably, the alkyl substituted furan is selected from 2 -methylfuran, 2,3-dimethylfuran, 2,4-dimethylfuran, 2,5-dimethylfuran (DMF), and combinations thereof.

Preferably, the starting material is 2,5-dimethylfuran (DMF) or 2,5-hexanedione (HD).

In step (b), the starting material is subjected to a cycloaddition reaction with a monoene in the absence of solvent and in the presence of the metal triflate catalyst to produce an alkyl substituted benzene.

For producing the alkyl substituted benzene at lower cost, a molar ratio of the metal triflate catalylst to the starting material ranging from 1:50 to 1: 100000. For producing the alkyl substituted benzene at higher yield, a molar ratio of the metal triflate catalylst to the starting material ranging from 1:5000 to 1: 30000. For allowing the starting material to contact and mix with the monoene in more effective manner, the starting material in step (b) is in a liquid state.

The monoene may include one or more C1 to C8 monoene. Preferably, the monoene is selected from the group consisting of ethylene, propene, 1-hexene, cyclohexene, and combinations thereof. More preferably, the monoene is ethylene.

The metal trilflate catalyst is selected from the group consisting of copper (II) trifluoromethanesulfonate (Cu(OTf)₂), zinc trifluoromethanesulfonate (Zn(OTf)₂), scandium trifluoromethanesulfonate (Sc(OTf)₂), yttrium trifluoromethanesulfonate (Y(OTf)₂), yttrium trifluoromethanesulfonate hydrate (Y(OTf)₂ hydrate), indium(III) trifluoromethanesulfonate (In(OTf)₂), and combinations thereof.

For allowing the starting material to contact and mix with the monoene in more effective manner, the cycloaddition reaction is conducted under a pressure ranging from 1000 psi to 2000 psi at a temperature ranging from 200° C. to 300° C.

In addition, a time period for the cycloaddition reaction is greater than 4 hours and not greater than 11 hours. Preferably, step (b) is implemented in two stages, i.e., an initial stage and a subsequent final stage. In the initial stage, the temperature is controlled above 200° C. and less than 270° C. for a time period ranging from 30 minutes to 60 minutes. In the subsequent final stage, the temperature is controlled at a range from 270° C. to 300° C. for a time period ranging from 4 hours to 10 hours.

The embodiments of the disclosure will now be explained in more detail below by way of the following examples and comparative examples.

### EXAMPLE 1 (EX 1)

A starting material (2,5-dimethylfuran, 90 g, about 100 ml) was placed in a high pressure reactor. Then, a metal triflate catalyst (copper (II) trifluoromethanesulfonate, 0.051 g) was added to the reactor, and nitrogen gas was introduced into the reactor to replace air for 3 times. Next, ethylene was introduced into the reactor at the room temperature to permit the pressure inside the reactor to reach to 520 psi. Thereafter, the temperature inside the reactor was raised to and kept at 250° C. for reaction for 0.5 hour, and then raised to and kept at 270° C. for reaction for 4.5 hours. During the above reactions, the pressure inside the reactor was gradually decreased from 1600 psi to 1200 psi. Finally, the temperature inside the reactor was cooled to room temperature, the pressure inside the reactor was relieved, and a yellowish-brown product including para-xylene was poured out of the reactor.

### EXAMPLES 2 to 11 (EX 2 to EX 11)

Products of Examples 2 to 11 were prepared according to the process employed for preparing the product of Example 1, except that the starting materials and/or the catalysts are different, as listed in the following Table 1.

### COMPARATIVE EXAMPLE 1 (CE 1)

A starting material (2,5-dimethylfuran, 8 g) and a solvent (tetrahydrofuran (THF), 221 ml) were placed in a high pressure reactor to have a total volume of about 230 ml. Then, copper (II) trifluoromethanesulfonate, (0.045 g) was added to the reactor, and nitrogen gas was introduced into the reactor to replace air for 3 times. Next, ethylene was introduced into the reactor at the room temperature to permit the pressure inside the reactor to reach to 520 psi. Thereafter, the temperature inside the reactor was raised to and kept at 270° C. for reaction for 5 hour. During the above reactions, the pressure inside the reactor was gradtially decreased from 1600 psi to 1200 psi. Finally, the temperature inside the reactor was cooled to room temperature, the pressure inside the reactor was relieved, and a yellowish-brown product including para-xylene was poured out of the reactor.

### COMPARATIVE EXAMPLES 2 to 13 (CE 2 to CE 13)

Products of Comparative Examples 2 to 13 were prepared according to the process employed for preparing the product of Comparative Example 1, except that the ratios of the starting material, the solvent, and/or the copper (II) trifluoromethanesulfonate are different, as listed in the following Table 2.

### COMPARATIVE EXAMPLE 14 (CE 14)

A starting material (2,5-dimethylfuran, 57.6 g) and a solvent (tetrahydrofuran (THF), 35 ml) were placed in a high pressure reactor to have a total volume of about 100 ml. Then, copper (II) trifluoromethanesulfonate, (0.007 g) was added to the reactor, and nitrogen gas was introduced into the reactor to replace air for 3 times. Next, ethylene was introduced into the reactor at the room temperature to permit the pressure inside the reactor to reach to 520 psi. Thereafter, the temperature inside the reactor was raised to and kept at 250° C. for reaction for 0.5 hour, and then raised to and kept at 270° C. for reaction for 4.5 hours. During the above reactions, the pressure inside the reactor was gradually decreased from 1600 psi to 1200 psi. Finally, the temperature inside the reactor was cooled to room temperature, the pressure inside the reactor was relieved, and a yellowish-brown product including para-xylene was poured out of the reactor.

Evaluations

Amounts of para-xylene obtained in the products of Examples 1 to 11 and Comparative Examples 1 to 14 were analyzed using a high performance liquid chromatography (HPLC) equipped with a diode array detector and a C18 column. A mobile phase for the HPLC was 0.05wt % phosphoric acid aqueous solution/acetonitrile, and a flow rate of the mobile phase was of 0.1 ml/min. Initially, the C18 column was eluated with 0.05wt % phosphoric acid aqueous solution. Then, the C18 column was further eluated with a mixture of 0.05wt % phosphoric acid aqueous solution and acetonitrile for a 30-minute period. During the 30-minute period, the percentage of acetonitrile in the mobile phase was gradually increased to 100%, and the percentage of 0.05wt % phosphoric acid aqueous solution was gradually decreased to 0%. Based on the obtained amounts of the para-xylene, yield, conversion, and selectivity for each of Examples 1 to 11 and Comparative Examples 1 to 14 were calculated based on the following equations (I) to (III), and are listed in Tables 1 and 2.

Yield of para-xylene=(moles of the obtained para-xylene/moles of the starting material before the reaction)×100% (I)

Conversion of the starting material=[1−(moles of the starting material after the reaction/moles of the staring material before the reaction)]×100% (II)

Selectivity for para-xylene=(yield of para-xylene/conversion of the starting material)×100% (III)

TABLE 1
(Examples, no solvent, total volume of the
starting material of 0.1 L)

| | Starting material | | Metal triflate catalyst | | | | | | |
|---|---|---|---|---|---|---|---|---|---|
| | | Weight | | Weight | mole | Yield | Conv. | Sele. | Amount |
| EX | Comp. | (g) | Comp. | (g) | % | (%) | (%) | (%) | (g) |
| 1 | DMF | 90 | Cu(OTf)₂ | 0.051 | 0.015 | 73 | 97 | 75 | 72.5 |
| 2 | | | | 0.034 | 0.01 | 78 | 97 | 80 | 77.5 |
| 3 | | | | 0.020 | 0.006 | 80 | 96 | 83 | 79.5 |
| 4 | | | | 0.010 | 0.003 | 80 | 95 | 84 | 79.5 |
| 5 | | | | 0.005 | 0.0015 | 74 | 91 | 81 | 73.5 |
| 6 | DMF | 90 | Zn(OTf)₂ | 0.010 | 0.003 | 81 | 94 | 86 | 80.5 |
| 7 | | 90 | Sc(OTf)₂ | 0.014 | | 77 | 94 | 82 | 76.5 |
| 8 | | 90 | Y(OTf)₂ | 0.015 | | 77 | 94 | 82 | 76.5 |
| 9 | | 90 | Y(OTf)₂ hydrate | 0.016 | | 75 | 94 | 80 | 74.5 |
| 10 | | 90 | In(OTf)₂ | 0.016 | | 80 | 96 | 83 | 79.5 |
| 11 | HD | 96 | Cu(OTf)₂ | 0.009 | 0.003 | 44 | 74 | 59 | 46.6 |

Note:
Comp. = component;
DMF = 2,5-dimethylfuran;
HD = 2,5-hexanedione;
mole % of catalyst = (moles of the catalyst/moles of the starting material) × 100%
Yield = Yield of para-xylene;
Conv. = Conversion of the starting material;
Sele. = Selectivity for para-xylene.
Amount = the obtained amount of para-xylene

TABLE 2
(Comparative Examples, with solvent)

| | DMF starting material | | THF | Total | Cu(OTf)₂ catalyst | | | | | |
|---|---|---|---|---|---|---|---|---|---|---|
| | Weight | Conc. | solvent | vol. | Weight | mole | Yield | Conv. | Sele. | Amount |
| CE | (g) | (M) | (mL) | (L) | (g) | % | (%) | (%) | (%) | (g) |
| 1 | 8 | 0.4 | 221 | 0.23 | 0.045 | 0.15 | 90 | 96 | 94 | 7.9 |
| 2 | | | | | 0.024 | 0.08 | 89 | 95 | 94 | 7.8 |
| 3 | | | | | 0.018 | 0.06 | 85 | 91 | 93 | 7.5 |
| 4 | | | | | 0.012 | 0.04 | 78 | 84 | 93 | 6.9 |
| 5 | 22 | 1 | 205 | 0.23 | 0.050 | 0.06 | 91 | 96 | 95 | 22.1 |
| 6 | 44 | 2 | 181 | | 0.066 | 0.04 | 87 | 94 | 93 | 42.3 |
| 7 | 66 | 3 | 156 | | 0.025 | 0.01 | 87 | 93 | 93 | 63.4 |
| 8 | 3.8 | 0.4 | 96 | 0.1 | 0.021 | 0.15 | 90 | 96 | 94 | 3.8 |
| 9 | 28.8 | 3 | 68 | | 0.022 | 0.02 | 90 | 95 | 95 | 28.6 |
| 10 | 38.4 | 4 | 57 | | 0.217 | 0.15 | 70 | 99 | 71 | 29.7 |
| 11 | 38.4 | 4 | 57 | | 0.022 | 0.015 | 90 | 96 | 94 | 38.2 |
| 12 | 48 | 5 | 46 | | 0.027 | 0.015 | 87 | 96 | 91 | 46.1 |
| 13 | 57.6 | 6 | 35 | | 0.022 | 0.01 | 83 | 94 | 88 | 52.8 |
| 14 | 57.6 | 6 | 35 | | 0.007 | 0.003 | 68 | 81 | 84 | 43.2 |

Note:
DMF = 2,5-dimethylfuran;
THF = tetrahydrofuran;
Total vol. = total volume of DMF and THF;
Conc. = molar concentration of DMF = moles of DMF/total vol.;
mole % is of catalyst = (moles of the catalyst/moles of the DMF starting material) × 100%
Yield = Yield of para-xylene;
Conv. = Conversion of the starting material;
Sele. = Selectivity for para-xylene.
Amount = the obtained amount of para-xylene

Please refer to entries 2 to 5 of Table 2 disclosed in U.S. Pat. No. 9,260,359 B2, the conversion of DMF and the selectivity for para-xylene are relatively low when para-xylene was obtained from the cycloaddition reaction between DMF and ethylene in the absence of solvent and in the presence of a Lewis acid of acetic anhydride, acetic acid, chloroacetic anhydride, or chloroacetic acid. The inventors of this application had found that when the Lewis acid used in the prior art was replaced by metal triflate catalyst, as shown in Table 1, the yield of para-xylene, the conversion of the starting material, and the selectivity for para-xylene can be greatly improved.

Furthermore, as shown in Tables 1 and 2, compared to Comparative Examples 1 to 14, the obtained amount of para-xylene in each of Examples 1 to 10 was relatively high (72.5 g-80.5 g). In other words, in the reactor with the same volume, alkyl substituted benzene (para-xylene) can be produced in greater amount by using the method of this application. In addition, the cycloaddition reaction between the starting material and monoene without solvent is environmental-friendly and cost-saving.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for producing alkyl substituted benzene, comprising the steps of:
(a) providing a starting material selecting from the group consisting of furan, an alkyl substituted furan, 2-methylfuran, 2,3-dimethylfuran, 2,4-dimethylfuran, 2,5-dimethylfuran, 2,5-hexanedione, and combinations thereof; and
(b) subjecting the starting material to a cycloaddition reaction with a monoene in the absence of solvent and in the presence of the metal triflate catalyst to produce an alkyl substituted benzene.

2. The method according to claim 1, wherein the metal trilflate catalyst is selected from the group consisting of copper (II) trifluoromethanesulfonate, zinc trifluoromethanesulfonate, scandium trifluoromethanesulfonate, yttrium trifluoromethanesulfonate, yttrium trifluoromethanesulfonate hydrate, indium(III) trifluoromethanesulfonate, and combinations thereof.

3. The method according to claim 2, wherein, instep (d), a molar ratio of the metal triflate catalylst to the starting material ranging from 1:50 to 1:100000.

4. The method according to claim 3, wherein, instep (d), a molar ratio of the metal triflate catalylst to the starting material ranging from 1:5000 to 1:30000.

5. The method according to claim 1, wherein the monoene is selected from the group consisting of ethylene, propene, 1-hexene, cyclohexene, and combinations thereof.

6. The method according to claim 1, wherein the starting material is 2,5-dimethylfuran or 2,5-hexanedione, and the monoene is ethylene.

7. The method according to claim 1, wherein the starting material in step (b) is in a liquid state.

8. The method according to claim 6, wherein the cycloaddition reaction is conducted under a pressure ranging from 1000 psi to 2000 psi at a temperature ranging from 200° C. to 300° C.

9. The method according to claim 8, wherein step (b) is implemented in two stages:
in an initial stage, the temperature is controlled above 200° C. and less than 270° C. for a time period ranging from 30 minutes to 60 minutes; and
in a subsequent final stage, the temperature is controlled at a range from 270° C. to 300° C. for a time period ranging from 4 hours to 10 hours.
